(19) United States
(12) Patent Application Publication
(10) Pub. No.: US 2022/0105297 A1
(43) Pub. Date: Apr. 7, 2022

(54) SYSTEM AND METHOD TO CREATE CUSTOM COLOR ADHESIVE SECUREMENT ARRANGEMENT

(71) Applicant: KONINKLIJKE PHILIPS N.V., Eindhoven (NL)

(72) Inventors: Lauren Patricia Chodkowski, Pittsburgh, PA (US); Richard Thomas Haibach, Pittsburgh, PA (US); Praveen Kumar Pandian Shanmuganathan, Pittsburgh, PA (US); Natasha Gilbert, Monroeville, PA (US)

(21) Appl. No.: 17/491,851

(22) Filed: Oct. 1, 2021

Related U.S. Application Data
(60) Provisional application No. 63/087,373, filed on Oct. 5, 2020.

(51) Int. Cl. A61M 16/06

(52) U.S. Cl. CPC A61M 16/0688; A61M 2207/00; A61M 2205/584; A61M 16/0666

(57) ABSTRACT

A method of creating a custom color adhesive securement arrangement for use with a patient interface includes performing a scan of a patient's face, extracting color values of a portion of the patient's face from the scan, determining one or more colors based on the extracted color values, obtaining an adhesive sheet having the one or more colors, and cutting the securement arrangement from the adhesive sheet.

### CROSS-REFERENCE TO RELATED APPLICATIONS

This patent application claims the priority benefit under 35 U.S.C. § 119(e) of U.S. Provisional Application No. 63/087,373, filed on Oct. 5, 2020, the contents of which are herein incorporated by reference.

### BACKGROUND OF THE INVENTION

### 1. Field of the Invention

The disclosed concept relates to adhesive arrangements, and more particularly to adhesive arrangements for adhering to the skin of a patient. The present invention further relates to adhesive arrangements for use in securing patient interfaces to the skin of a patient.

### 2. Description of the Related Art

Many individuals suffer from disordered breathing during sleep. Sleep apnea is a common example of such sleep disordered breathing suffered by millions of people throughout the world. One type of sleep apnea is obstructive sleep apnea (OSA), which is a condition in which sleep is repeatedly interrupted by an inability to breathe due to an obstruction of the airway, typically the upper airway or pharyngeal area. Obstruction of the airway is generally believed to be due, at least in part, to a general relaxation of the muscles which stabilize the upper airway segment, thereby allowing the tissues to collapse the airway. Another type of sleep apnea syndrome is a central apnea, which is a cessation of respiration due to the absence of respiratory signals from the brain's respiratory center. An apnea condition, whether obstructive, central, or mixed, which is a combination of obstructive and central, is defined as the complete or near cessation of breathing, for example a 90% or greater reduction in peak respiratory airflow.

Those afflicted with sleep apnea experience sleep fragmentation and complete or nearly complete cessation of ventilation intermittently during sleep with potentially severe degrees of oxyhemoglobin desaturation. These symptoms may be translated clinically into extreme daytime sleepiness, cardiac arrhythmias, pulmonary-artery hypertension, congestive heart failure and/or cognitive dysfunction. Other consequences of sleep apnea include right ventricular dysfunction, carbon dioxide retention during wakefulness, as well as during sleep, and continuous reduced arterial oxygen tension. Sleep apnea sufferers may be at risk for excessive mortality from these factors as well as by an elevated risk for accidents while driving and/or operating potentially dangerous equipment.

Even if a patient does not suffer from a complete or nearly complete obstruction of the airway, it is also known that adverse effects, such as arousals from sleep, can occur where there is only a partial obstruction of the airway. Partial obstruction of the airway typically results in shallow breathing referred to as a hypopnea. A hypopnea is typically defined as a 50% or greater reduction in the peak respiratory airflow. Other types of sleep disordered breathing include, without limitation, upper airway resistance syndrome (UARS) and vibration of the airway, such as vibration of the pharyngeal wall, commonly referred to as snoring.

It is well known to treat sleep disordered breathing by applying a continuous positive air pressure (CPAP) to the patient's airway. This positive pressure effectively “splints” the airway, thereby maintaining an open passage to the lungs. It is also known to provide a positive pressure therapy in which the pressure of gas delivered to the patient varies with the patient's breathing cycle, or varies with the patient's breathing effort, to increase the comfort to the patient. This pressure support technique is referred to as bi-level pressure support, in which the inspiratory positive airway pressure (IPAP) delivered to the patient is higher than the expiratory positive airway pressure (EPAP). It is further known to provide a positive pressure therapy in which the pressure is automatically adjusted based on the detected conditions of the patient, such as whether the patient is experiencing an apnea and/or hypopnea. This pressure support technique is referred to as an auto-titration type of pressure support, because the pressure support device seeks to provide a pressure to the patient that is only as high as necessary to treat the disordered breathing.

Pressure support therapies as just described involve the placement of a patient interface device including a mask component having a soft, flexible sealing cushion member on the face of the patient. The mask component may be, without limitation, a nasal mask that covers the patient's nose, a nasal/oral mask that covers the patient's nose and mouth, or a full face mask that covers the patient's face. Such patient interface devices may also employ other patient contacting components, such as forehead supports, cheek pads and chin pads. The patient interface device is connected to a gas delivery tube or conduit and interfaces the pressure support device with the airway of the patient, so that a flow of breathing gas can be delivered from the pressure/flow generating device to the airway of the patient.

Traditionally, such patient interface devices have been secured to the face/head of the patient by a headgear component having one or more straps which wrap around all, or a portion, of the patient's head. Recently, adhesive arrangements have been employed, either in-whole, or in-part, to secure patient interface devices to the face of a patient.

It is important for a patient to comply with usage of a patient interface device. While comfort is a primary reason why a patient does not comply with patient interface device usage, aesthetics are also a reason why a patient may not comply with patient interface device usage. Aesthetic design is a concern in patient interface device designs.

### SUMMARY OF THE INVENTION

Accordingly, as one aspect of the disclosed concept, a method of creating a custom color adhesive securement arrangement for use with a patient interface comprises: performing a scan of a patient's face; extracting color values of a portion of the patient's face from the scan; determining one or more colors based on the extracted color values; obtaining an adhesive sheet having the one or more colors; and cutting the securement arrangement from the adhesive sheet.

As another aspect of the disclosed concept, a system for creating a custom color adhesive securement arrangement for use with a patient interface comprises: a processing unit including: a color extraction unit structured to receive a scan of a patient's face and to extract color values of a portion of the patient's face from the scan; and a color matching unit structured to determine one or more colors based on the extracted color values and to output information indicative of the one or more colors.

As another aspect of the disclosed concept, a system for creating a custom color adhesive securement arrangement for use with a patient interface comprises: a processing unit including: a color extraction unit structured to receive a scan of a patient's face and to extract color values of a portion of the patient's face from the scan; and a color matching unit structured to determine a single color based on the extracted color values, to select an adhesive sheet from a plurality of premade adhesive sheets of different colors, wherein the selected adhesive sheet has a color most closely matching the single color among the plurality of premade adhesive sheets, and to output information indicative of the selected adhesive sheet.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIG. 1 is a view of an airway pressure support system in accordance with an example embodiment of the disclosed concept;

FIG. 2 is a view of the pressure support system of FIG. 1 worn by a patient in accordance with an example embodiment of the disclosed concept;

FIG. 3 is a view of a substrate from which an adhesive portion of a patient interface is cut in accordance with an example embodiment of the disclosed concept;

FIG. 4 is a schematic diagram of a system for creating a custom color securement arrangement for use with a patient interface in accordance with an example embodiment of the disclosed concept;

FIG. 5 is a flowchart of a method of creating a custom color adhesive securement arrangement for use with a patient interface in accordance with an example embodiment of the disclosed concept.

### DETAILED DESCRIPTION OF EXEMPLARY EMBODIMENTS

As required, detailed embodiments of the present invention are disclosed herein. However, it is to be understood that the disclosed example embodiments described herein are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are coupled directly in contact with each other. As used herein, “fixedly coupled” or “fixed” means that two components are coupled so as to move as one while maintaining a constant orientation relative to each other.

As used herein, the statement that two or more parts or components “engage” one another shall mean that the parts exert a force against one another either directly or through one or more intermediate parts or components. As used herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

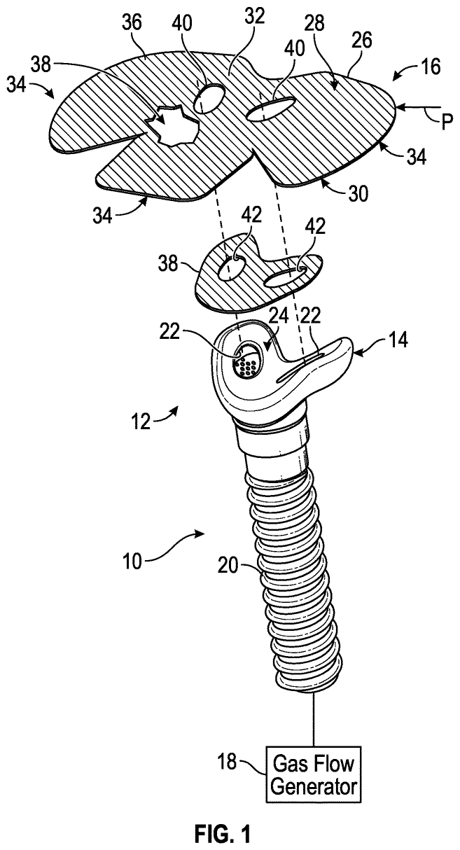

FIG. 1 is a simplified perspective front view of an airway pressure support system 10 including a patient interface assembly 12 having a patient interface 14 for use in providing a flow of a breathing gas to the airway of a patient (not shown) which may utilize a securement arrangement 16, in accordance with one or more example embodiments of the present invention, for securing patient interface 14 to a patient. Airway pressure support system 10 includes a gas flow generator 18 (shown schematically) and a hose 20 (partially shown schematically) having a first end (not numbered) coupled to gas flow generator 18 and an opposite second end (not numbered) coupled to patient interface 14. Gas flow generator 18 is structured to generate a flow of breathing gas to be delivered to an airway of a patient via hose 20 and patient interface 14.

In the example arrangement shown in FIG. 1, patient interface 14 is in the form of a cradle-like nasal interface for generally engaging the underside of the nose of a patient. Accordingly, patient interface 14 includes a pair nasal apertures 22 defined in a contoured surface 24. In one example embodiment of the present invention, contoured surface 24 and each nasal aperture 22 is custom dimensioned/formed based on facial dimensions of a particular patient. Such custom dimensioning/forming may readily be carried out using 3D scanning and printing techniques known in the art. It is to be appreciated that other arrangements of patient interface 12 may be employed without varying from the scope of the present invention.

Continuing to refer to FIG. 1, securement arrangement 16 is provided for securing patient interface 14 to the face of a patient. Securement arrangement 16 may be mass produced or custom created for the dimensions of a particular patient and is formed from a generally thin (e.g., having a thickness of at least 0.04 mm), substantially planar, substrate material 26 having a first planar surface 28, which faces toward a patient, and a second planar surface 30, opposite first planar surface 28, which faces toward patient interface 14. Planar substrate material 26 may generally be any pliable material such as, for example, without limitation, foam, silicone polyurethane, latex, or any other suitable material. In the example shown in FIG. 1, planar substrate material 26 includes a central portion 32 having a number of tabs 34 projecting outward therefrom, with each of tabs 34 structured to wrap up around, and be secured to the patient's nose. It is to be appreciated that planar substrate material may be of other shapes and/or dimensions without varying from the scope of the present invention.

Securement arrangement 16 further includes an adhesive material 36 (shown schematically as hatching in FIG. 1, e.g., without limitation, a silicone or acrylic based adhesive layer or any other suitable adhesive layer) provided on first planar surface 28 for use in adhering adhesive arrangement 16 to the skin of a patient. A removable layer of a release film 38 (only a portion of which is shown in FIG. 1) may be provided covering adhesive material 36 for shielding adhesive material 36 until desired to be exposed and adhered to the skin of a patient.

In example system 10 illustrated in FIG. 1, securement arrangement 16 is structured to be positioned generally between patient interface 14 and the skin of the patient. More particularly, securement arrangement 16 is structured to be positioned between contoured surface 24 of patient interface 14 and the skin of the patient and secured to contoured surface 24 via a secondary adhesive arrangement 38 (e.g., a single layer material having upper and lower adhesive surfaces integral therewith or applied thereto) which may be disposed directly on second planar surface 30 of substrate material 26, or alternatively, as a separate element, such as shown in the example of FIG. 1. Additionally, secondary adhesive arrangement 38 may include a foam core for providing dampening resulting from torque due to hose 20. In order to provide for a reliable seal about each nare of the patient, and to allow for the passage of the flow of breathing gas from each of nasal apertures 22 of patient interface 14 to the respective nares of the patient, each of substrate material 26, and similarly secondary adhesive arrangement 38, respectively includes a pair of apertures 40 and 42, respectively, defined therein of similar, and preferably identical shape (e.g., to minimize turbulence, maximize comfort, etc.), as nasal apertures 22.

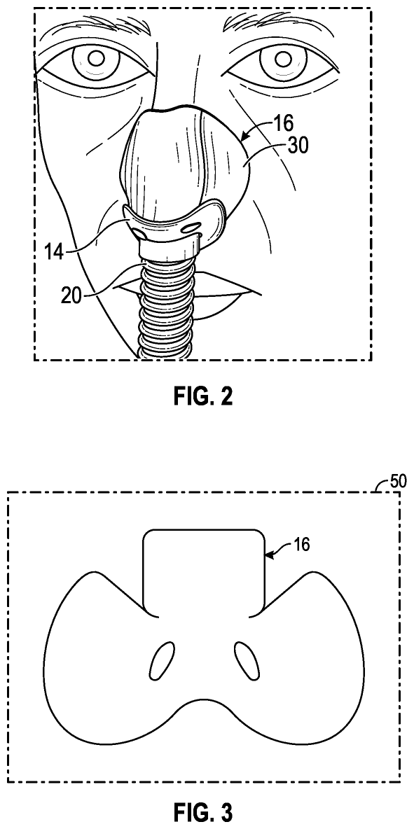

In FIG. 2, securement arrangement 16 is shown in use, adhered to a patient's nose. Securement arrangement 16 is coupled to patient interface 14 via secondary adhesive arrangement 38 (hidden in FIG. 2). As shown in FIG. 2, second planar surface 38 (which is now folded to correspond to the shape of the patient's nose) is prominently visible. As will be described in more detail herein, in accordance with an example embodiment of the disclosed concept, the color of second planar surface 38 of securement arrangement 16 is made to match the color of the nostril region of the patient. This makes the appearance of securement arrangement 16 subtler and more aesthetically pleasing, thus making it more likely that the patient will comply with usage of pressure support system 10. A default color of the second planar surface 38 may be a plain white, which does not match the patient's skin and would visually stand out when securement arrangement 16 is worn. This issue would only be amplified with patients with darker complexion.

In accordance with an example embodiment, securement arrangement 16 is cut from a sheet 50 of substrate material, such as sheet 50 shown in FIG. 3. In the example embodiment shown in FIG. 3, one securement arrangement 16 is shown on sheet 50. However, it will be appreciated that multiple securement arrangements 16 may be formed from a single sheet 50.

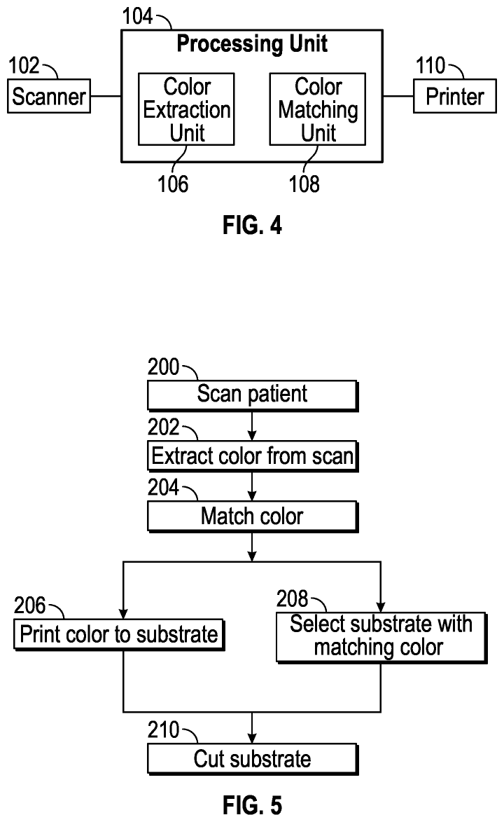

FIG. 4 is a schematic diagram of a system 100 for creating a custom color securement arrangement for use with a patient interface in accordance with an example embodiment of the disclosed concept. System 100 includes a scanner 102, a processing unit 104, and a printer 110.

Scanner 102 is structured to obtain a 3D scan of a portion of a patient.

Scanner 102 may be one of a various number of devices capable of obtaining a 3D scan of a patient's face. Any suitable method (e.g., without limitation, structured lighting, stereo, time-of-flight (ToF), etc.) may be used to obtain a 3D scan of a patient's face. The 3D scan will subsequently be used to determine the color for securement arrangement 16.

Processing unit 104 may include a processor and a memory. The processor that may be, for example and without limitation, a microprocessor, a microcontroller, or some other suitable processing device or circuitry, that interfaces with the memory. The memory can be any of one or more of a variety of types of internal and/or external storage media such as, without limitation, RAM, ROM, EPROM(s), EEPROM(s), FLASH, and the like that provide a storage register, i.e., a machine readable medium, for data storage such as in the fashion of an internal storage area of a computer, and can be volatile memory or nonvolatile memory. Various routines may be stored in the memory and, when executed by the processor, may cause the process to implement corresponding functions.

Processing unit 104 is structured to receive the 3D scan of the patient from scanner 102. Processing unit 104 may include a color extraction unit 106 and a color matching unit 108. Color extraction unit 106 is structured to extract the color in the localized region of the patient's nostril. Color extraction unit 106 may include a nostril classifier that localizes the nostril region of the patient in the 3D scan and extracts color values in the nostril region from the 3D scan data.

Color matching unit 108 is structured to determine a color for the securement arrangement 16 based on the extracted color values. For example, color matching unit 108 may determine a single color corresponding to the nostril region of the patient. The single color may be determined, for example, by taking an average or median of the colors in the nostril region of the patient. The determined color will match the color of the patient's skin in the nostril region. Color matching unit 108 may also output the determined color in a format suitable for use by printer 110.

In some example embodiments, color matching unit 108 may create a color map instead of determining a single color. The color map may map colors extracted from the 3D scan to corresponding points on securement arrangement 16. In this manner, an exact mapping of the colors of the patient's nostril region may be applied to securement arrangement 16. In some example embodiments, further feature extraction may be employed to neutralize variations such as freckles or blemishes. For example, rather than adding a freckle to the color map, the freckle may be neutralized and the color map may instead replace the freckle with a color corresponding to the area around the freckle. In this manner, the resultant coloration of securement arrangement 16 will match the color of the patient's skin, but without selected blemishes.

Color matching unit 108 is structured to output the determined color to printer 110. Printer 110 is structured to print the determined color on a sheet of substrate in the region where securement arrangement 16 will be cut from. In embodiments where a color map is employed, the color map may be output to printer 110.

Processing unit 104 may output additional information to printer 110. For example, processing unit 104 may output additional information to printer 110 such as ID numbers and location indicators for robotic alignment. Printer 110 may also print this additional information on the sheet from which securement arrangement 16 will be cut.

In some example embodiments, a plurality of sheets from which securement arrangement 16 may be cut may be premade in a variety of different colors. Rather than outputting color information to printer 110, color matching unit 108 may match the determined color to one of the premade sheets most closely matching the determined color. Securement arrangement 16 may then be cut from the selected sheet.

FIG. 5 is a flowchart of a method of creating a custom color adhesive securement arrangement for use with a patient interface in accordance with an example embodiment of the disclosed concept. The method may be employed in system 100, for example.

The method begins at 200 by scanning a patient to obtain a 3D scan of the patient. Then, at 202, color of the patient is extracted from the 3D scan. The color may be extracted from a localized region of the patient, such as the nostril region. Color values corresponding to the nostril region may be extracted. At 204, the extracted color values are matched to a color to be used for the securement arrangement. In one example, a single color is determined as the color to be used. In another example, a color map including multiple colors is generated.

Once the color is matched, the method may proceed to 206 or 208. In one embodiment, the method proceeds to 206 where the determined color or color map is printed onto a substrate, such as sheet 50 (shown in FIG. 3) in the region where the securement arrangement will be cut from. Additional information, such as ID numbers or location indicators may also be printed to the substrate. In an embodiment, the method may instead proceed from 204 to 208 where a substrate with a color most closely matching the determined color is selected. In this example, a plurality of substrates of different colors may be premade and the one most closely matching the determined color is selected. In some examples, additional information, such as ID numbers and location indicators, may be printed to the selected substrate.

From 206 or 208, the method proceeds to 210, where the securement arrangement is cut from the substrate. Subsequently, the securement arrangement may be folded and adhered to the patient as is shown for example in FIG. 2. In example embodiments, at least the surface opposite the adhesive side of the securement arrangement (the side facing outward from the patient) will have a color that matches the color of the patient's skin. This will create a visual effect of the securement arrangement blending with the patient's skin tone and thus having a more subtle appearance than if it were a color that contrasted with the patient's skin tone.

From the foregoing, it is thus to be appreciated that the disclosed concept provides for adhesive arrangements that may be alternated so as to reduce skin irritations commonly caused by conventional arrangements. Combinations of such arrangements may be provided in common packaging (e.g., a box or other suitable commercial packaging) as a kit (e.g., without limitation, a week/month supply) for an end user. Although described herein as being used for securing patient interface 14 to a patient, it is to be appreciated that securements arrangements such as described herein as well and the general concepts related thereto may be readily applied to other applications of adhering elements to the skin of a patient without varying from the scope of the present invention.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

What is claimed is:

1. A method of creating a custom color adhesive securement arrangement for use with a patient interface, the method comprising:
performing a scan of a patient's face;
extracting color values of a portion of the patient's face from the scan;
determining one or more colors based on the extracted color values;
obtaining an adhesive sheet having the one or more colors; and
cutting the securement arrangement from the adhesive sheet.

2. The method of claim 1, wherein the one or more colors is a single color determined based on an average or median of the color values of the portion of the patient's face.

3. The method of claim 1, wherein the one or more colors is a plurality of colors, and wherein the method further comprises generating a color map to map the color values of the portion of the patient's face to corresponding points on the securement arrangement.

4. The method of claim 3, further comprising neutralizing variations in color values due to freckles or blemishes on the patient when generating the color map.

5. The method of claim 1, wherein obtaining the adhesive sheet having the one or more colors includes printing the one or more colors on the adhesive sheet.

6. The method of claim 5, wherein the one or more colors are printed on the adhesive sheet in a region from which the securement arrangement is cut.

7. The method of claim 5, further comprising printing additional information including at least one of an identification number and a location identifier on the adhesive substrate.

8. The method of claim 1, wherein the one or more colors is a single color, wherein obtaining the adhesive sheet includes selecting the adhesive sheet from a plurality of premade adhesive sheets of different colors, wherein the selected adhesive sheet has a color most closely matching the single color among the plurality of premade adhesive sheets.

9. The method of claim 1, wherein the portion of the patient's face from which color values are extracted is a nostril region of the patient.

10. The method of claim 1, wherein the adhesive sheet has an adhesive side and a non-adhesive side, and wherein the one or more colors are on the non-adhesive side.

11. The method of claim 1, wherein the securement arrangement is structured to adhere to the patient's face and be disposed between the patient's face and the patient interface.

12. A system for creating a custom color adhesive securement arrangement for use with a patient interface, the system comprising:
a processing unit including:
a color extraction unit structured to receive a scan of a patient's face and to extract color values of a portion of the patient's face from the scan; and
a color matching unit structured to determine one or more colors based on the extracted color values and to output information indicative of the one or more colors.

13. The system of claim 12, further comprising:
a printer structured to receive the information indicative of the one or more colors and to print the one or more colors on an adhesive sheet from which the securement arrangement will be cut.

14. The system of claim 12, further comprising a scanner structured to generate the scan of the patient's face.

15. A system for creating a custom color adhesive securement arrangement for use with a patient interface, the system comprising:
a processing unit including:
a color extraction unit structured to receive a scan of a patient's face and to extract color values of a portion of the patient's face from the scan; and
a color matching unit structured to determine a single color based on the extracted color values, to select an adhesive sheet from a plurality of premade adhesive sheets of different colors, wherein the selected adhesive sheet has a color most closely matching the single color among the plurality of premade adhesive sheets, and to output information indicative of the selected adhesive sheet.
